(19) United States
(12) Patent Application Publication
(10) Pub. No.: US 2013/0096277 A1
(43) Pub. Date: Apr. 18, 2013

(54) PROCESSES AND INTERMEDIATES

(71) Applicant: Vertex Pharmaceuticals Incorporated, Cambridge, MA (US)

(72) Inventor: GERALD J. TANOURY, MALBOROUGH, MA (US)

(73) Assignee: VERTEX PHARMACEUTICALS INCORPORATED, CAMBRIDGE, MA (US)

(21) Appl. No.: 13/692,565

(22) Filed: Dec. 3, 2012

Related U.S. Application Data
(63) Continuation of application No. PCT/US2011/039049, filed on Jun. 3, 2011.
(60) Provisional application No. 61/486,130, filed on May 13, 2011.
(60) Provisional application No. 61/351,054, filed on Jun. 3, 2010.

(51) Int. Cl. C07K 5/107; C07D 498/10; C07D 209/52

(52) U.S. Cl. CPC C07K 5/1016; C07D 209/52; C07D 498/10
USPC 530/330; 548/515; 548/409; 548/240

(57) ABSTRACT

A process for preparing enantioselectively a compound of formula I-1a or I-1b:

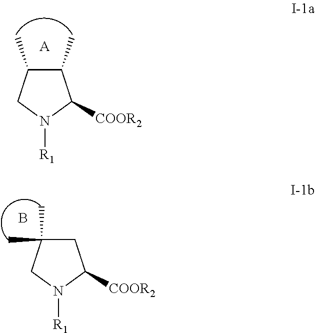

over a compound of formulas I-2-I-7:

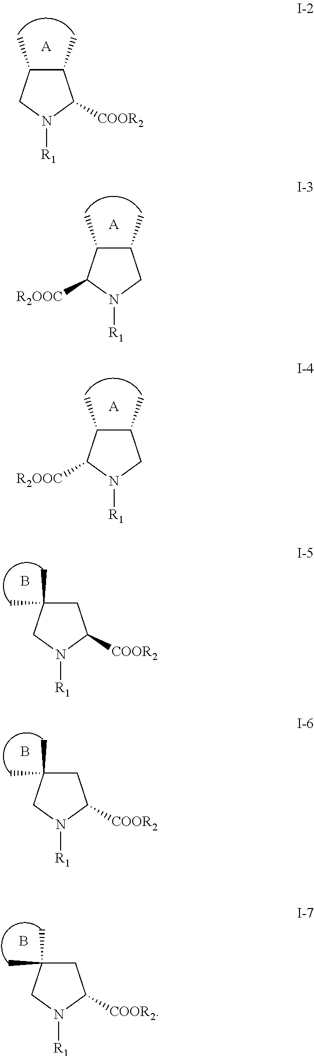

### PRIORITY CLAIM

This application claims priority to U.S. provisional application Ser. No. 61/351,054, filed on Jun. 6, 2010, and to U.S. provisional application Ser. No. 61/486,130, filed on May 13, 2011. The entire contents of both priority applications are incorporated herein by reference.

### FIELD OF THE INVENTION

This invention relates to processes and intermediates for the preparation of protease inhibitors, in particular, serine protease inhibitors.

### BACKGROUND OF THE INVENTION

Infection by hepatitis C virus (“HCV”) is a compelling human medical problem. HCV is recognized as the causative agent for most cases of non-A and non-B hepatitis, with an estimated human sero-prevalence of 3% globally (A. Alberti et al., “Natural History of Hepatitis C,” J. Hepatology, 31 (Suppl. 1), pp. 17-24 (1999)). Nearly four million individuals may be infected in the United States alone. (M. J. Alter et al., “The Epidemiology of Viral Hepatitis in the United States,” Gastroenterol. Clin. North Am., 23, pp. 437-455 (1994); M. J. Alter “Hepatitis C Virus Infection in the United States,” J. Hepatology, 31 (Suppl. 1), pp. 88-91 (1999)).

Upon first exposure to HCV, only about 20% of infected individuals develop acute clinical hepatitis, while others appear to resolve the infection spontaneously. In almost 70% of instances, however, the virus establishes a chronic infection that may persist for decades. (S. Iwarson, “The Natural Course of Chronic Hepatitis,” FEMS Microbiology Reviews, 14, pp. 201-204 (1994); D. Lavanchy, “Global Surveillance and Control of Hepatitis C,” J. Viral Hepatitis, 6, pp. 35-47 (1999)). Prolonged chronic infection can result in recurrent and progressively worsening liver inflammation, which often leads to more severe disease states such as cirrhosis and hepatocellular carcinoma. (M. C. Kew, “Hepatitis C and Hepatocellular Carcinoma,” FEMS Microbiology Reviews, 14, pp. 211-220 (1994); I. Saito et. al., “Hepatitis C Virus Infection is Associated with the Development of Hepatocellular Carcinoma,” Proc. Natl. Acad. Sci., 87, pp. 6547-6549 (1990)). Unfortunately, there are no broadly effective treatments for the debilitating progression of chronic HCV.

Protease inhibitors, and in particular serine protease inhibitors, are useful in the treatment of HCV infections, as disclosed in WO 02/18369. WO 02/18369 also discloses processes and intermediates for the preparation of these compounds. These processes lead to racemization of certain steric carbon centers. See, e.g., pages 223-22. As a result, a need remains for enantioselective processes for the preparation of these compounds.

### SUMMARY OF THE INVENTION

In one aspect, the invention provides processes and intermediates for producing bicyclic derivatives of formula I-1a or I-1b, which are useful in producing protease inhibitors.

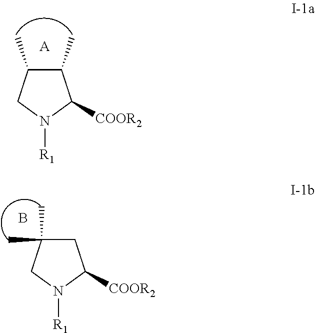

In formulas I-1a and I-1b,
Ring A is a C₃₋₁₂ cycloaliphatic ring;
Ring B is a C₃₋₁₂ heterocycloaliphatic ring containing an additional 0 to 2 hetero atoms, each independently selected from O, N, and S, wherein ring B can be optionally substituted with 1 to 4 groups, each independently selected from alkyl, halo, alkoxy, aryl, and hydroxyl;
R₁ is H or a protecting group; and
R₂ is H, a protecting group, or C₁₋₁₂ aliphatic.

One embodiment is a process for preparing enantioselectively compounds of formula I-1a or I-1b over compounds of formulas I-2-I-7:

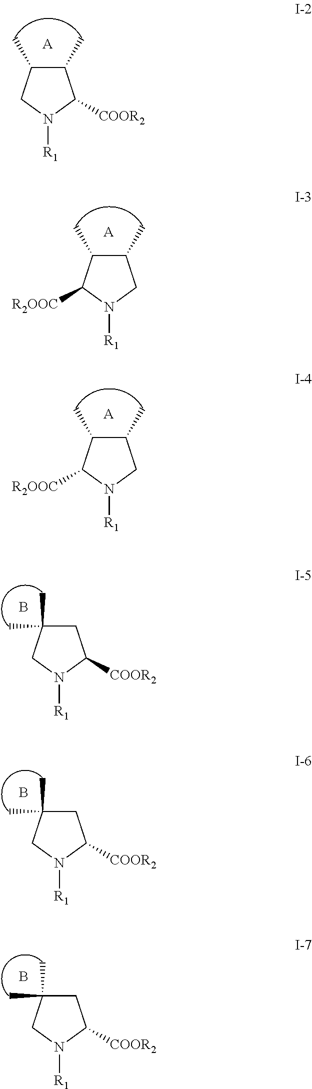

The process comprises the step of carboxylating a compound of formulas II-a or II-b:

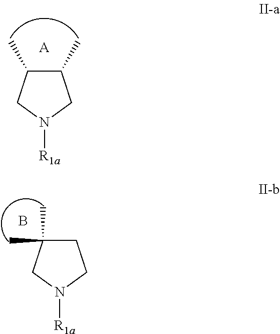

wherein R₁ₐ is a protecting group, in the presence of a compound of formula III:

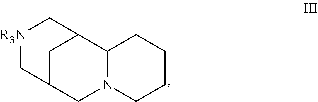

wherein R₃ is C₁₋₁₂ aliphatic.

In one embodiment, ring A is a C₃₋₆ cycloaliphatic ring.

In one embodiment, ring A is cyclopropyl.

In one embodiment, ring A is cyclopentyl.

In one embodiment, ring A is 1,1-dimethylcyclopropyl.

In one embodiment, ring A is:

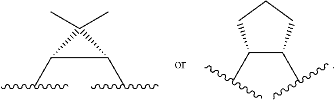

In one embodiment, ring A is

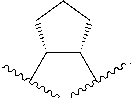 [SMILES: *[C@@H]1CCC[C@@H]1*]

In one embodiment, ring A is

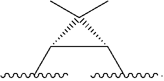 [SMILES: *[C@@H]1[C@H](*)C1(C)C]

In one embodiment, ring B is a 5-membered heterocyclic ring.

In one embodiment, ring B is an optionally substituted ring of the following formula:

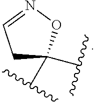

In one embodiment, ring B is substituted with an aryl ring optionally substituted with 1 to 4 groups, each independently selected from alkyl, halo, alkoxy, and hydroxyl.

In one embodiment, ring B is aryl. In another embodiment, the aryl ring is phenyl.

Further, in another embodiment, the aryl ring is:

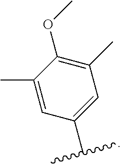

In yet another embodiment, ring B is:

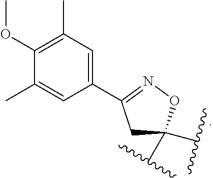

In one embodiment, R₂ is H. In another embodiment, R₂ is C₁₋₁₂ aliphatic. In yet another embodiment, R₂ is tert-butyl.

In one embodiment, the step of carboxylating a compound of formula II-a or II-b is in the presence of a compound of formula III-a:

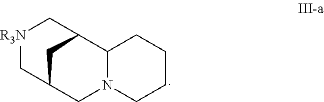

In one embodiment, the step of carboxylating a compound of formula II-a or II-b is in the presence of a compound of formula III-b:

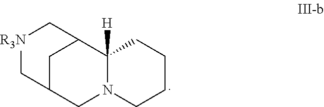

In one embodiment, the step of carboxylating a compound of formula II-a or II-b is in the presence of a compound of formula III-c:

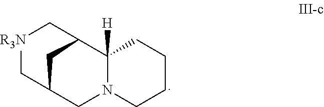

In one embodiment, R₃ is C₁₋₁₂ aliphatic.

More particularly, R₃ is C₁₋₆ alkyl.

In one embodiment, R₃ is C₁₋₆ cycloalkyl.

Further, in another embodiment, R₃ is selected from the group consisting of methyl, ethyl, n-propyl, iso-propyl, iso-butyl, tert-butyl, n-butyl, n-pentyl, and iso-pentyl.

More particularly, R₃ is tert-butyl.

In another embodiment, R₃ is iso-butyl.

In one embodiment, R₁ₐ is tert-butyl carbamate (Boc).

In one embodiment, the carboxylation step includes treating a compound of formula II-a or II-b with carbon dioxide and a lithium base in the presence of an aprotic solvent.

In one embodiment, the aprotic solvent is selected from toluene, ethyl acetate, benzene, and methyl tert-butyl ether (MTBE). In another embodiment, the aprotic solvent is MTBE.

In one embodiment, the lithium base is sec-butyl lithium.

In one embodiment, the process of the present invention gives rise to a mixture of products including I-1a (exo), I-3 (exo), I-2 (endo), and I-4 (endo).

In one embodiment, the process of the present invention includes the combined weight percent in a mixture comprising compounds of formula I-1a and I-3 (the exo-isomers) and compounds of formula I-2 and I-4 (the endo-isomers) is 100 weight percent

In another embodiment, the ratio of the combined weight percent of I-1a and I-3 (exo-isomers) to that of I-2 and I-4 (endo-isomers) is at least 60 to 40.

In one embodiment, the exo/endo ratio is at least 60 to 40.

In one embodiment, the exo/endo ratio is at least 80 to 20.

In one embodiment, the exo/endo ratio is at least 90 to 10.

In one embodiment, the exo/endo ratio is at least 95 to 5.

In one embodiment, the exo/endo ratio is at least 97 to 3.

In one embodiment, the process of the present invention further comprises removing at least a portion of the compounds of formula I-2 and/or I-4 from the product mixture.

In one embodiment, removing I-2 and/or I-4 comprises crystallizing the compound of formula I-1a or I-1b.

In one embodiment, removing I-2 and/or I-4 comprises recrystallizing the compound of formula I-1a or I-1b.

In one embodiment, the ratio of the weight percent of I-1a to I-3 is at least 60 to 40. In one embodiment, the ratio of the weight percent of I-1a to I-3 is at least 80 to 20. In one embodiment, the ratio of the weight percent of I-1a to I-3 is at least 90 to 10. In one embodiment, the ratio of the weight percent of I-1a to I-3 is at least 95 to 5. In one embodiment, the ratio of the weight percent of I-1a to I-3 is at least 99 to 1. In one embodiment, the ratio of the weight percent of I-1a to I-3 is at least 99.6 to 0.4. In one embodiment, the ratio of the weight percent of I-1a to I-3 is at least 100 to 0.

Another aspect of the present invention is a process for preparing a compound of formula 10:

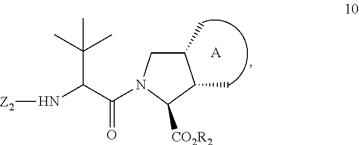

wherein Z₂ is H or a protecting group, and R₂ is H, a protecting group, or C₁₋₁₂ aliphatic; comprising the steps of:
i) providing a compound of formula II-a:

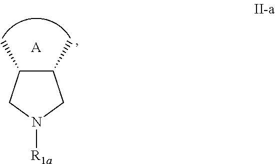

wherein R₁ₐ is a protecting group, and ring A is C₃₋₁₂ cycloaliphatic;
ii) forming a 2-anion of the compound of formula II-a in the presence of a compound of formula III:

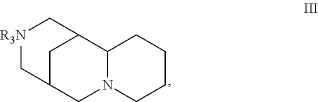

wherein R₃ is C₁₋₁₂ aliphatic or a protecting group;
iii) treating the anion of step ii) with carbon dioxide to produce enantioselectively a compound of formula I-1a;
iv) reacting the compound of formula I-1a, wherein R₁ is H, with a compound of formula 26:

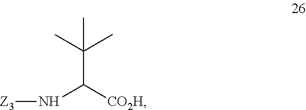

wherein Z₃ is a protecting group.

Another aspect of the present invention is a process for preparing a compound of formula 10:

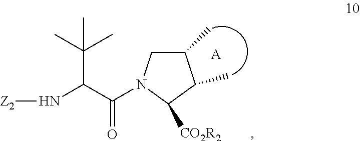

wherein Z₂ is H or a protecting group, and R₂ is H, a protecting group, or C₁₋₁₂ aliphatic; comprising the steps of:
i) forming a 2-anion of the compound of formula II-a:

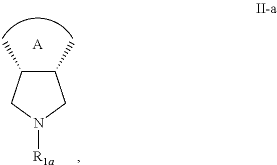

wherein R₁ₐ is a protecting group, and ring A is C₃₋₁₂ cycloaliphatic;
in the presence of a compound of formula III:

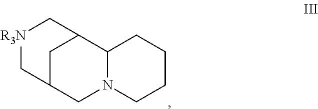

wherein R₃ is C₁₋₁₂ aliphatic or a protecting group;
ii) treating the anion of step i) with carbon dioxide to produce enantioselectively a compound of formula I-1a;
iii) reacting the compound of formula I-1a, wherein R₁ is H, with a compound of formula 26:

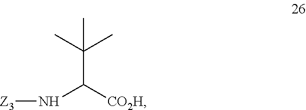

wherein Z₃ is a protecting group.

In one embodiment, R₃ is tert-butyl.

In one embodiment, the step of carboxylating a compound of formula II-a or II-b is in presence of the compound of formula III-a:

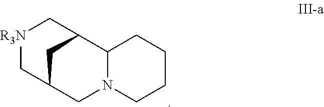

In one embodiment, the step of carboxylating a compound of formula II-a or II-b is in presence of the compound of formula III-b:

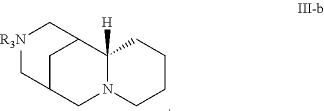

In one embodiment, the step of carboxylating a compound of formula II-a or II-b is in presence of the compound of formula III-c:

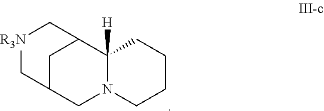

In one embodiment, R₃ is C₃₋₁₂ aliphatic. In another embodiment, R₃ is a cycloaliphatic. Further, in another embodiment, R₃ is C₁₋₆ aliphatic. In yet another embodiment, R₃ is C₁₋₆ alkyl.

In another embodiment, R₃ is methyl, ethyl, n-propyl, iso-propyl, iso-butyl, n-butyl, n-pentyl, or iso-pentyl. In yet another embodiment, R₃ is iso-butyl.

In one embodiment, ring A is:

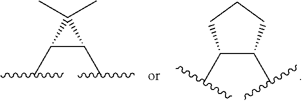

In one embodiment, ring A is

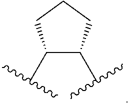 [SMILES: *[C@@H]1CCC[C@@H]1*]

In one embodiment, the compound of formula 26 is the compound of formula 26-a:

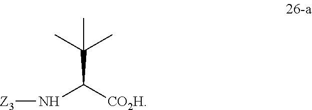

In one embodiment, ring A is

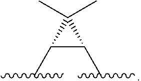 [SMILES: *[C@@H]1[C@H](*)C1(C)C]

In one embodiment, the compound of formula 26 is the compound of formula 26-b:

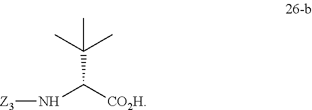

In one embodiment, the compound of formula 10 is the compound of formula 10-a:

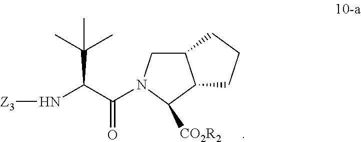

In one embodiment, the compound of formula 10 is a compound of formula 10-a, wherein Z₂ is H, and R₂ is ten-butyl.

In one embodiment, the compound of formula 10 is the compound of formula 10-b:

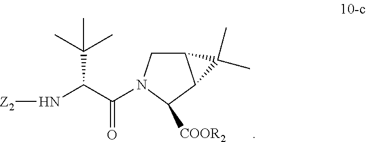

In one embodiment, the compound of formula 10 is a compound of formula 10-b, wherein Z₂ is H, and R₂ is tert-butyl.

One aspect of the present invention is a compound of formula I-1a(1) made by the processes disclosed herein:

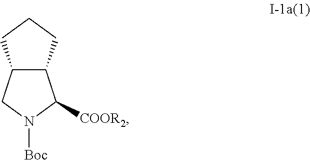

wherein R₂ is H, a protecting group, or C₁₋₁₂ aliphatic.

One aspect of the present invention is a compound of formula I-1a(2) made by the processes disclosed herein:

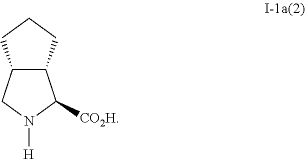 [SMILES: O=C(O)[C@H]1NC[C@@H]2CCC[C@@H]21]

Another aspect of the present invention is a compound of formula I-1a(3) made by the processes disclosed herein:

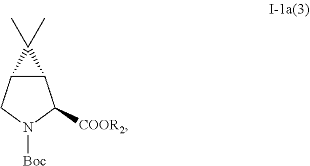

wherein R₂ is H, a protecting group, or C₁₋₁₂ aliphatic.

One aspect of the present invention is a compound of formula I-1a(4) made by the processes disclosed herein:

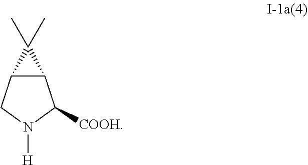

One aspect of the present invention is a compound of formula 10-a made by the processes disclosed herein:

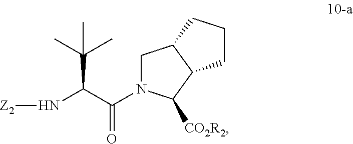

wherein Z₂ is H or a protecting group, and R₂ is H, a protecting group, or C₁₋₁₂ aliphatic.

One aspect of the present invention is a compound of formula 10-b made by the processes disclosed herein:

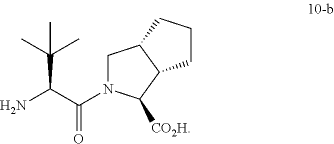 [SMILES: CC(C)(C)[C@H](N)C(=O)N1C[C@@H]2CCC[C@@H]2[C@H]1C(=O)O]

One aspect of the present invention is a compound of formula 10-c made by the processes disclosed herein:

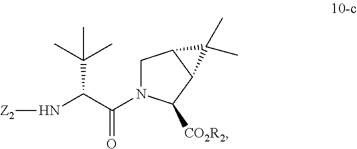

wherein Za is H or a protecting group, and R₂ is H, a protecting group, or C₁₋₁₂ aliphatic.

One aspect of the present invention is a compound of formula 10-d made by the processes disclosed herein:

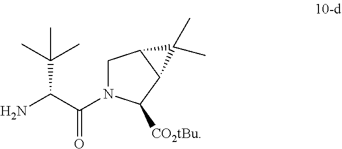 [SMILES: CC(C)(C)OC(=O)[C@@H]1[C@@H]2[C@H](CN1C(=O)[C@H](N)C(C)(C)C)C2(C)C]

### DETAILED DESCRIPTION OF THE INVENTION

### Definitions

For the purposes of this invention, the chemical elements are identified in accordance with the Periodic Table of the Elements, CAS version, Handbook of Chemistry and Physics, 75^{th} Ed. Additionally, general principles of organic chemistry are described by Thomas Sorrell in Organic Chemistry, University Science Books, Sausalito (1999), and by M. B. Smith and J. March in Advanced Organic Chemistry, 5^{th} Ed., John Wiley & Sons, New York (2001), both of which are hereby incorporated by reference.

As described herein, compounds of the invention may be optionally substituted with one or more substituents, such as are illustrated generally above, or as exemplified by particular classes, subclasses, and species of the invention.

It must be noted that as used herein and in the claims, the singular forms “a,” “an,” and “the” include plural referents unless the context clearly dictates otherwise. Thus, for example, reference to “a binder” includes two or more binders, and reference to “a pharmaceutical agent” includes two or more pharmaceutical agents, and so forth.

As used herein, the term “compound” refers to the compound(s) that are defined by structural formulas respectively drawn herein. Furthermore, unless otherwise stated, the term “compound” can include a salt of the compound(s).

As used herein, the term “aliphatic” encompasses the terms alkyl, alkenyl, alkynyl, and cycloaliphatic, each of which is optionally substituted as set forth below.

As used herein, an “alkyl” group refers to a saturated aliphatic hydrocarbon group containing 1-8 (e.g., 1-6 or 1-4) carbon atoms. An alkyl group can be straight, cyclic, or branched. Examples of alkyl groups include, but are not limited to, methyl, ethyl, propyl, isopropyl, butyl, isobutyl, sec-butyl, ten-butyl, n-pentyl, n-heptyl, or 2-ethylhexyl. An alkyl group can be substituted (i.e., optionally substituted) with one or more substituents selected from the group which consists of halo, cycloaliphatic (e.g., cycloalkyl or cycloalkenyl), heterocycloaliphatic (e.g., heterocycloalkyl or heterocycloalkenyl), aryl, heteroaryl, alkoxy, aroyl, heteroaroyl, acyl (e.g., (aliphatic)carbonyl, (cycloaliphatic)carbonyl, or (heterocycloaliphatic)carbonyl), nitro, cyano, amido (e.g., (cycloalkylalkyl)carbonylamino, arylcarbonylamino, aralkylcarbonylamino, (heterocycloalkyl)carbonylamino, (heterocycloalkylallyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl), amino (e.g., aliphaticamino, cycloaliphaticamino, or heterocycloaliphaticamino), sulfonyl (e.g., aliphatic-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, and hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, and haloalkyl.

As used herein, an “alkenyl” group refers to an aliphatic carbon group that contains 2-8 (e.g., 2-6 or 2-4) carbon atoms and at least one double bond. Like an alkyl group, an alkenyl group can be straight or branched. Examples of an alkenyl group include, but are not limited to, allyl, isoprenyl, 2-butenyl, and 2-hexenyl. An alkenyl group can be optionally substituted with one or more substituents such as halo, cycloaliphatic (e.g., cycloalkyl or cycloalkenyl), heterocycloaliphatic (e.g., heterocycloalkyl or heterocycloalkenyl), aryl, heteroaryl, alkoxy, aroyl, heteroaroyl, acyl (e.g., (aliphatic)carbonyl, (cycloaliphatic)carbonyl, or (heterocycloaliphatic)carbonyl), nitro, cyano, amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl), amino (e.g., aliphaticamino, cycloaliphaticamino, heterocycloaliphaticamino, or aliphaticsulfonylamino), sulfonyl (e.g., alkyl-SO₂—, cycloaliphatic-SO₂—, or aryl-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, and hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, and haloalkenyl.

As used herein, an “alkynyl” group refers to an aliphatic carbon group that contains 2-8 (e.g., 2-6 or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl (e.g., aliphaticsulfanyl or cycloaliphaticsulfanyl), sulfinyl (e.g., aliphaticsulfinyl or cycloaliphaticsulfinyl), sulfonyl (e.g., aliphatic-SO₂—, aliphaticamino-SO₂—, or cycloaliphatic-SO₂—), amido (e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino or heteroarylaminocarbonyl), urea, thiourea, sulfamoyl, sulfamide, alkoxycarbonyl, alkylcarbonyloxy, cycloaliphatic, heterocycloaliphatic, aryl, heteroaryl, acyl (e.g., (cycloaliphatic)carbonyl or (heterocycloaliphatic)carbonyl), amino (e.g., aliphaticamino), sulfoxy, oxo, carboxy, carbamoyl, (cycloaliphatic)oxy, (heterocycloaliphatic)oxy, and (heteroaryl)alkoxy.

As used herein, an “amido” encompasses both “aminocarbonyl” and “carbonylamino.” These terms, when used alone or in connection with another group, refer to an amido group such as —N(R^{X})—C(O)—R^{Y} or —C(O)—N(R^{X})₂, when used terminally, and they refer to an amide group such as —C(O)—N(R^{X})— or —N(R^{X})—C(O)— when used internally, wherein R^{X} and R^{Y} are defined below. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, and cycloalkylamido.

As used herein, an “amino” group refers to —NR^{X}R^{Y}, wherein each of R^{X} and R^{Y} is independently selected from hydrogen, aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, and (heteroaraliphatic)carbonyl, each of which being defined herein and is optionally substituted. Examples of amino groups include alkylamino, dialkylamino, and arylamino. When the term “amino” is not the terminal group (e.g., alkylcarbonylamino), it is represented by —NR^{X}—. R^{X} has the same meaning as defined above.

As used herein, an “aryl” group, used alone or as part of a larger moiety as in “aralkyl”, “aralkoxy”, or “aryloxyalkyl,” refers to monocyclic (e.g., phenyl), bicyclic (e.g., indenyl, naphthalenyl, tetrahydronaphthyl, tetrahydroindenyl), and tricyclic (e.g., fluorenyl tetrahydrofluorenyl, or tetrahydroanthracenyl, anthracenyl) ring systems in which the monocyclic ring system is aromatic or at least one of the rings in a bicyclic or tricyclic ring system is aromatic. The bicyclic and tricyclic groups include benzofused 2- to 3-membered carbocyclic rings. For example, a benzofused group includes phenyl fused with two or more C₄₋₈ carbocyclic moieties. An aryl is optionally substituted with one or more substituents, such as aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, oxo (on a non-aromatic carbocyclic ring of a benzofused bicyclic or tricyclic aryl), nitro, carboxy, amido, acyl (e.g., aliphaticcarbonyl, (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl), sulfonyl (e.g., aliphatic-SO₂— or amino-SO₂—), sulfinyl (e.g., aliphatic-S(O)— or cycloaliphatic-S(O)—), sulfanyl (e.g., aliphatic-S—), cyano, halo, hydroxy, mercapto, sulfoxy, urea, thiourea, sulfamoyl, sulfamide, and carbamoyl. Alternatively, an aryl can be unsubstituted.

Non-limiting examples of substituted aryls include haloaryl (e.g., mono-, di- (such as p,m-dihaloaryl), or (trihalo)aryl), (carboxy)aryl (e.g., (alkoxycarbonyl)aryl, ((aralkyl)carbonyloxy)aryl, or (alkoxycarbonyl)aryl), (amido)aryl (e.g., (aminocarbonyl)aryl, (((alkylamino)alkyl)aminocarbonyl)aryl, (alkylcarbonyl)aminoaryl, (arylaminocarbonyl)aryl, or (((heteroaryl)amino)carbonyl)aryl), aminoaryl (e.g., ((alkylsulfonyl)amino)aryl or ((dialkyl)amino)aryl), (cyanoalkyl)aryl, (alkoxy)aryl, (sulfamoyl)aryl (e.g., (aminosulfonyl)aryl), (alkylsulfonyl)aryl, (cyano)aryl, (hydroxyalkyl)aryl, ((alkoxy)alkyl)aryl, (hydroxy)aryl, ((carboxy)alkyl)aryl, (((dialkyl)amino)alkyl)aryl, (nitroalkyl)aryl, (((alkylsulfonyl)amino)alkyl)aryl, ((heterocycloaliphatic)carbonyl)aryl, ((alkylsulfonyl)alkyl)aryl, (cyanoalkyl)aryl, (hydroxyalkyl)aryl, (alkylcarbonyl)aryl, alkylaryl, (trihaloalkyl)aryl, p-amino-m-alkoxycarbonylaryl, p-amino-m-cyanoaryl, p-halo-m-aminoaryl, and (m-(heterocycloaliphatic)-o-(alkyl))aryl.

As used herein, an “araliphatic” group, such as “aralkyl,” refers to an aliphatic group (e.g., a C₁₋₄ alkyl group) that is substituted with an aryl group. Aliphatic, alkyl, and aryl are defined herein. An example of araliphatic such as an aralkyl group is benzyl.

As used herein, an “aralkyl” group refers to an alkyl group (e.g., a C₁₋₄ alkyl group) that is substituted with an aryl group. Both alkyl and aryl have been defined above. An example of an aralkyl group is benzyl. An aralkyl is optionally substituted with one or more substituents such as aliphatic (e.g., substituted or unsubstituted alkyl, alkenyl, or alkynyl, including carboxyalkyl, hydroxyalkyl, or haloalkyl, such as trifluoromethyl), cycloaliphatic (e.g., substituted or unsubstituted cycloalkyl or cycloalkenyl), (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido (e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino), cyano, halo, hydroxy, acyl, mercapto, alkylsulfanyl, sulfoxy, urea, thiourea, sulfamoyl, sulfamide, oxo, and carbamoyl.

As used herein, a “bicyclic ring system” includes 8- to 12- (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which being optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2]decyl, bicyclo[2.2.2]octyl, adamantyl, azacycloalkyl, and ((aminocarbonyl)cycloalkyl)cycloalkyl. A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, and bicyclo[3.3.1]nonenyl. A cycloalkyl or cycloalkenyl group can be optionally substituted with one or more substituents such as aliphatic (e.g., alkyl, alkenyl, or alkynyl), cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or ((heteroaraliphatic)carbonylamino), nitro, carboxy (e.g., HOOC—, alkoxycarbonyl, or alkylcarbonyloxy), acyl (e.g., (cycloaliphatic)carbonyl, (cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl), cyano, halo, hydroxy, mercapto, sulfonyl (e.g., alkyl-SO₂—, or aryl-SO₂—), sulfinyl (e.g., alkyl-S(O)—), sulfanyl (e.g., alkyl-S—), sulfoxy, urea, thiourea, sulfamoyl, sulfamide, oxo, and carbamoyl.

As used herein, “cyclic moiety” includes cycloaliphatic, heterocycloaliphatic, aryl, or heteroaryl, each of which has been defined previously.

As used herein, the term “heterocycloaliphatic” encompasses a heterocycloalkyl group and a heterocycloalkenyl group, each of which being optionally substituted as set forth below.

As used herein, a “heterocycloalkyl” group refers to a 3-10 membered mono- or bicyclic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A monocyclic heterocycloalkyl group can be fused with a phenyl moiety such as tetrahydroisoquinoline. A “heterocycloalkenyl” group, as used herein, refers to a mono- or bicyclic (e.g., 5- to 10-membered mono- or bicyclic) non-aromatic ring structure having one or more double bonds, and wherein one or more of the ring atoms is a heteroatom (e.g., N, O, or S). Monocyclic and bicycloheteroaliphatics are numbered according to standard chemical nomenclature.

A heterocycloalkyl or heterocycloalkenyl group can be optionally substituted with one or more substituents such as aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, (cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, or (heteroaraliphatic)carbonylamino), nitro, carboxy HOOC—, alkoxycarbonyl, or alkylcarbonyloxy), acyl (e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl), nitro, cyano, halo, hydroxy, mercapto, sulfonyl (e.g., alkylsulfonyl or arylsulfonyl), sulfinyl (e.g., alkylsulfinyl), sulfanyl (e.g., alkylsulfanyl), sulfoxy, urea, thiourea, sulfamoyl, sulfamide, oxo, and carbamoyl.

As used herein, a “heteroaryl” group refers to a monocyclic, bicyclic, or tricyclic ring system having 4 to 15 ring atoms, wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, phthalazyl, quinazolyl, quinoxalyl, isoquinolyl, 4H-quinolizyl, benzo-1,2,5-thiadiazolyl, and 1,8-naphthyridyl.

Without limitation, monocyclic heteroaryls include furyl, thiophenyl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, and 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, and pteridyl. Bicyclic heteroaryls are numbered according to standard chemical nomenclature.

A heteroaryl is optionally substituted with one or more substituents such as aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, oxo (on a non-aromatic carbocyclic or heterocyclic ring of a bicyclic or tricyclic heteroaryl), carboxy, amido, acyl (e.g., aliphaticcarbonyl, (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl), sulfonyl (e.g., aliphaticsulfonyl or aminosulfonyl), sulfinyl (e.g., aliphaticsulfinyl), sulfanyl (e.g., aliphaticsulfanyl), nitro, cyano, halo, hydroxy, mercapto, sulfoxy, urea, thiourea, sulfamoyl, sulfamide, and carbamoyl. Alternatively, a heteroaryl can be =substituted.

Non-limiting examples of substituted heteroaryls include (halo)heteroaryl (e.g., mono- and di-(halo)heteroaryl), (carboxy)heteroaryl (e.g., (alkoxycarbonyl)heteroaryl), cyanoheteroaryl, aminoheteroaryl (e.g., ((alkylsulfonyl)amino)heteroaryl and ((dialkyl)amino)heteroaryl), (amido)heteroaryl (e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, or ((alkylcarbonyl)amino)heteroaryl), (cyanoalkyl)heteroaryl, (alkoxy)heteroaryl, (sulfamoyl)heteroaryl (e.g., (aminosulfonyl)heteroaryl), (sulfonyl)heteroaryl ((e.g., (alkylsulfonyl)heteroaryl), (hydroxyalkyl)heteroaryl, (alkoxyalkyl)heteroaryl, (hydroxy)heteroaryl, ((carboxy)alkyl)heteroaryl, (((dialkyl)amino)alkyl)heteroaryl, (heterocycloaliphatic)heteroaryl, (cycloaliphatic)heteroaryl, (nitroalkyl)heteroaryl, (((alkylsulfonyl)amino)alkyl)heteroaryl, ((alkylsulfonyl)alkyl)heteroaryl, (cyanoalkyl)heteroaryl, (acyl)heteroaryl (e.g., (alkylcarbonyl)heteroaryl), (alkyl)heteroaryl, and (haloalkyl)heteroaryl (e.g., trihaloalkylheteroaryl).

As used herein, “heteroaraliphatic” (such as a heteroaralkyl group) refers to an aliphatic group (e.g., a C₁₋₄ alkyl group) that is substituted with a heteroaryl group. Aliphatic, alkyl, and heteroaryl have been defined above.

As used herein, a “heteroaralkyl” group refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents, such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, and carbamoyl.

As used herein, an “acyl” group refers to a formyl group or R^{X}—C(O)— (such as alkyl-C(O)—, also referred to as “alkylcarbonyl”), wherein R^{X} and alkyl have been defined previously. Acetyl and pivaloyl are examples of acyl groups.

As used herein, an “aroyl” or “heteroaroyl” refers to an aryl-C(O)— or a heteroaryl-C(O)—. The aryl and heteroaryl portion of the aroyl or heteroaroyl are optionally substituted as previously defined.

As used herein, an “alkoxy” group refers to an alkyl-O— group, wherein alkyl has been defined previously.

As used herein, a “carbamoyl” group refers to a group having the structure —O—CO—NR^{X}R^{Y} or —NR^{X}—CO—O—R^{Z}, wherein R^{X} and R^{Y} have been defined above, and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, or —OC(O)R^{X} when used terminally and —OC(O)— or —C(O)O— when used internally.

As used herein, a “haloaliphatic” group refers to an aliphatic group substituted with 1-3 halogens. For example, the term haloalkyl includes the group —CF₃.

As used herein, a “mercapto” group refers to —SH.

As used herein, a “sulfo” group refers to —SO₃H or —SO₃R^{X} when used terminally and —S(O)₃-when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfonamide” group refers to the structure —S(O)₂—NR^{X}R^{Y} or —NR^{X}—S(O)₂—R^{Z} when used terminally and —S(O)₂—NR^{X}— or —NR^{X}—S(O)₂— when used internally, wherein R^{X}, R^{Y}, and R^{Z} have been defined above.

As used herein, a “sulfanyl” group refers to —S—R^{X} when used terminally and —S— when used internally, wherein R^{X} has been defined above. Examples of sulfanyl include aliphatic-S—, cycloaliphatic-S—, and aryl-S—, or the like.

As used herein a “sulfinyl” group refers to —S(O)—R^{X} when used terminally and —S(O)—when used internally, wherein R^{X} has been defined above. Examples of sulfinyl groups include aliphatic-S(O)—, aryl-S(O)—, (cycloaliphatic(aliphatic))-S(O)—, cycloalkyl-S(O)—, heterocycloaliphatic-S(O)—, and heteroaryl-S(O)—, or the like.

As used herein, a “sulfonyl” group refers to —S(O)₂—R^{X} when used terminally and —S(O)₂-when used internally, wherein R^{X} has been defined above. Exemplary sulfonyl groups include aliphatic-S(O)₂—, aryl-S(O)₂—, ((cycloaliphatic(aliphatic))-S(O)₂—, cycloaliphatic-S(O)₂—, heterocycloaliphatic-S(O)₂—, heteroaryl-S(O)₂—, and (cycloaliphatic(amido(aliphatic)))-S(O)₂—, or the like.

As used herein, a “sulfoxy” group refers to —O—SO—R^{X} or —SO—O—R^{X} when used terminally and —O—S(O)— or —S(O)—O— when used internally, wherein R^{X} has been defined above.

As used herein, a “halogen” or “halo” group refers to fluorine, chlorine, bromine, or iodine.

As used herein, an “alkoxycarbonyl” group, which is encompassed by “carboxy,” used alone or in combination with another group, refers to a group such as alkyl-O—C(O)—.

As used herein, an “alkoxyalkyl” group refers to an alkyl group such as alkyl-O-alkyl-, wherein alkyl has been defined above.

As used herein, a “carbonyl” group refers to —C(O)—.

As used herein, an “oxo” group refers to ═O.

As used herein, an “aminoalkyl” group refers to the structure (R^{X})₂N-alkyl-.

As used herein, a “cyanoalkyl” group refers to the structure (NC)-alkyl-.

As used herein, a “urea” group refers to the structure —NR^{X}—CO—NR^{Y}R^{Z}, and a “thiourea” group refers to the structure —NR^{X}—CS—NR^{Y}R^{Z} when used terminally and —NR^{X}—CO—NR^{Y}— or —NR^{X}—CS—NR^{Y}— when used internally, wherein R^{X}, R^{Y}, and R^{Z} have been defined above.

As used herein, a “guanidine” group refers to the structure N═C(N(R^{X}R^{Y}))N(R^{X}R^{Y}) or —NR^{X}—C(═NR^{X})NR^{X}R^{Y}, wherein R^{X} and R^{Y} have been defined above.

As used herein, an “amidino” group refers to the structure —C═(NR^{X})N(R^{X}R_{Y}), wherein R^{X} and R^{Y} have been defined above.

As used herein, the term “vicinal” refers to the placement of substituents on a group that includes two or more carbon atoms, wherein the substituents are attached to adjacent carbon atoms.

As used herein, the term “geminal” refers to the placement of substituents on a group that includes two or more carbon atoms, wherein the substituents are attached to the same carbon atom.

As used herein, the terms “terminally” and “internally” refer to the location of a group within a substituent. A group is terminal when the group is present at the end of the substituent and not further bonded to the rest of the chemical structure. Carboxyalkyl, i.e., R^{X}O(O)C-alkyl, is an example of a carboxy group used terminally. A group is internal when it is not terminal. Alkylcarboxy (e.g., alkyl-C(O)—O— or alkyl-O—C(O)—) and alkylcarboxyaryl (e.g., alkyl-C(O)—O-aryl- or alkyl-O—C(O)-aryl-) are examples of carboxy groups used internally.

As used herein, a “cyclic” group includes mono-, bi-, and tri-cyclic ring systems, such as cycloaliphatic, heterocycloaliphatic, aryl, and heteroaryl, each of which has been defined above.

As used herein, a “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxabicyclo[2.2.2]octyl, 1-azabicyclo[2.2.2]octyl, 3-azabicyclo[3.2.1]octyl, and 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, and carbamoyl.

As used herein, an “aliphatic chain” refers to a branched or straight aliphatic group (e.g., alkyl groups, alkenyl groups, or alkynyl groups). A straight aliphatic chain has the structure —(CH₂)ᵥ—, where v is 1-6. A branched aliphatic chain is a straight aliphatic chain that is substituted with one or more aliphatic groups. A branched aliphatic chain has the structure —(CHQ)ᵥ—, where v is 1-6 and Q is hydrogen or an aliphatic group; however, Q shall be an aliphatic group in at least one instance. The term aliphatic chain includes alkyl chains, alkenyl chains, and alkynyl chains, where alkyl, alkenyl, and alkynyl are defined above.

As used herein, the phrase “optionally substituted” is used interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R₁, R₂, and R₃, as well as other variables, encompass specific groups, such as alkyl and aryl. Unless otherwise noted, each of the specific groups for the variables R₁, R₂, and R₃, and other variables contained therein can be optionally substituted with one or more substituents described herein. Each substituent of a specific group is further optionally substituted with one to three of halo, cyano, oxo, alkoxy, hydroxy, amino, nitro, aryl, cycloaliphatic, heterocycloaliphatic, heteroaryl, haloalkyl, and alkyl. For instance, an alkyl group can be substituted with alkylsulfanyl, and the alkylsulfanyl can be optionally substituted with one to three of halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkoxy groups can form a ring together with the atom(s) to which they are bound.

As used herein, the term “substituted,” whether preceded by the term optionally or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, i.e., both rings share one common atom. Combinations of substituents envisioned by this invention are those combinations that result in the formation of stable or chemically feasible compounds.

As used herein, the phrase “stable or chemically feasible” refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, the phrase “preparing enantioselectively” refers to asymmetric synthetic preparation of enantiomerically-enriched compounds. This is further defined as the use of one or more techniques to prepare the desired compound in high enantiomeric excess (i.e., 60% or more). The techniques encompassed may include the use of chiral starting materials (e.g., chiral pool synthesis), the use of chiral auxiliaries and chiral catalysts, and the application of asymmetric induction.

As used herein, “enantiomeric excess” or “e.e.,” refers to the optical purity of a compound.

As used herein, “endo:exo” refers to the ratio of endo-isomers to exo-isomers.

As used herein, “enantiomeric ratio,” or “e.r.,” is the ratio of the percentage of one enantiomer in a mixture to that of the other.

As used herein, a “protecting group” is defined as a group that is introduced into a molecule to modify a functional group present in a molecule to prevent it from reacting in a subsequent chemical reaction and thus obtain chemoselectivity. It is removed from the molecule at a later step in the synthesis. For example, a carbobenzyloxy (Cbz) group can replace the hydrogen on an amine to prevent it from reacting with an electrophile, then the Cbz group can be removed by hydrolysis in a later step.

Acid and amine protecting groups as used herein are known in the art (see, e.g., T. W. Greene & P. G. M. Wutz, “Protective Groups in Organic Synthesis,” 3^{rd} Edition, John Wiley & Sons, Inc. (1999)). Examples of suitable protecting groups for acids include tert-butoxy, benzyloxy, allyloxy, and methoxymethoxy. Examples of suitable protecting groups for amines include 9-fluorenylmethyl carbamate, tert-butyl carbamate, benzyl carbamate, trifluoroacetamide, and p-toluenesulfonamide.

As used herein, an “effective amount” is defined as the amount required to confer a therapeutic effect on the treated patient and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, New York, 537 (1970). As used herein, “patient” refers to a mammal, including a human.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon, are within the scope of this invention. Such compounds are useful, for example, as analytical tools or probes in biological assays.

As used herein, “EDC” is 1-(3-dimethylaminopropyl)-3-ethylcarbodiimide, “HOBt” is 1-hydroxybenzotriazole, “THF” is tetrahydrofuran, “Cbz” is benzyloxycarbonyl, “DCM” is dichloromethane, and “Boc” is tert-butoxycarbonyl.

As used herein, “¹H NMR” stands for proton nuclear magnetic resonance, and “TLC” stands for thin layer chromatography.

### Processes and Intermediates

In one embodiment, the invention provides a process and intermediates for preparing a compound of formula I-1a as outlined in Scheme I, wherein R₁, R₁ₐ, R₂, R₃, and ring A are previously defined.

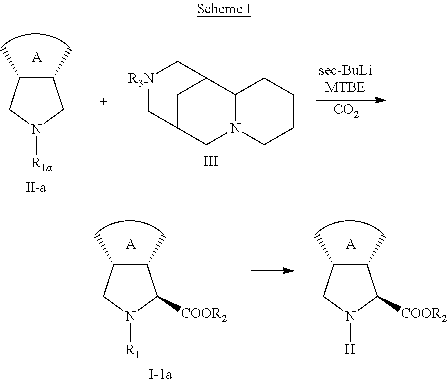

Carboxylation of the compound of formula II-a is achieved by first forming a 2-anion of formula II-a in the presence of a ligand, i.e., a compound of formula III. For formation of similar anions, see, e.g., Daniel. J. Pippel, et. al., J. Org. Chem., 1998, 63, 2; Donald J. Gallagher et al., J. Org. Chem., 1995, 60(22), 7092-7093; Shawn T. Kerrick et al., J. Am. Chem. Soc., 1991, 113(25), 9708-9710; Donald J. Gallagher et al., J. Org. Chem., 1995, 60(25), 8148-8154; and Peter Beak et al., J. Am. Chem. Soc., 1994, 116(8), 3231-3239. The 2-anion of formula II-a (not shown in Scheme I) is prepared by treatment of compound of formula II-a with a strong lithium base (e.g., sec-butyllithium or isopropyllithium) in the presence of a complexing agent (e.g., tetramethylethylenediamine, tetraethylethylenediamine, tetramethyl-1,2-cyclohexyldiamine, or 3,7-dipropyl-3,7-diazabicyclo[3.3.1]nonane) in a suitable aprotic solvent (e.g., tert-butylmethyl ether, diethylether, or toluene).

An optically active complexing agent of formula III can induce enantioselective carboxylation to give a product having an enantiomeric excess (e.e.) of from about 10% to about 95% (see, e.g., Beak et al., J. Org. Chem., 1995, 60, 8148-8154). In the presence of formula III, a compound of formula II-a can be treated with carbon dioxide to give a mixture of exo/endo compounds of formula I-1a, wherein the exo/endo ratio is 60 to 40, 80 to 20, 90 to 10, 95 to 5, or greater than 98 to 2.

Referring to Scheme I, a compound of formula II-a, wherein R₁ₐ is, e.g., tert-butoxycarbonyl (Boc), is prepared using known methods. See, e.g., T. W. Greene and P. G. M. Wuts, Protective Groups in Organic Synthesis, 3^{rd} edition, John Wiley and Sons, Inc. (1999).

In one embodiment, the invention provides a process and intermediates for preparing a ligand of formula III, as shown on Schemes II-A, II-B, and II-C. In Scheme II-A, R is an C₁₋₄ unbranched aliphatic. In Scheme II-B, R is H. In Scheme II-C, R is an alpha-branched aliphatic (e.g. iso-propyl).

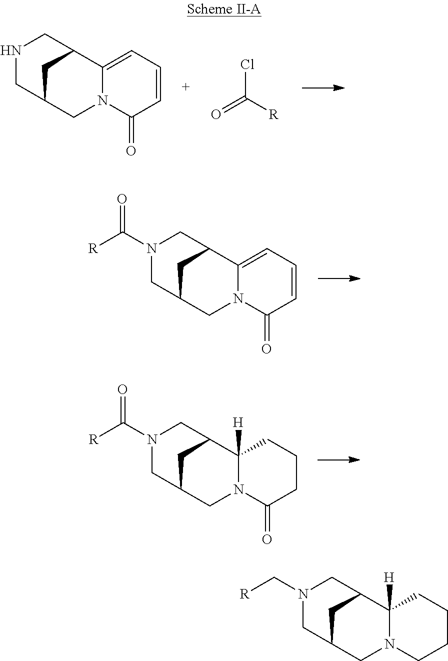

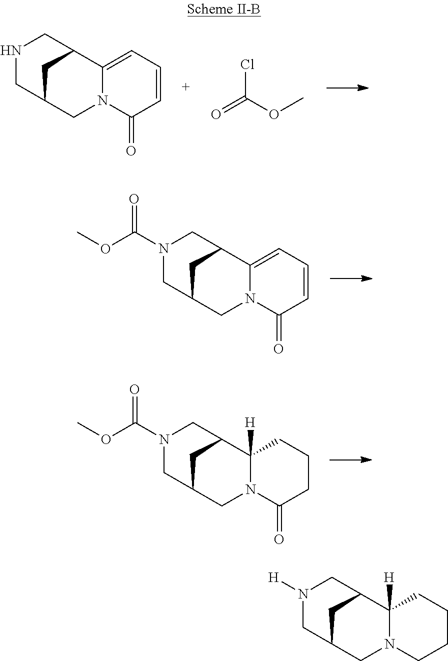

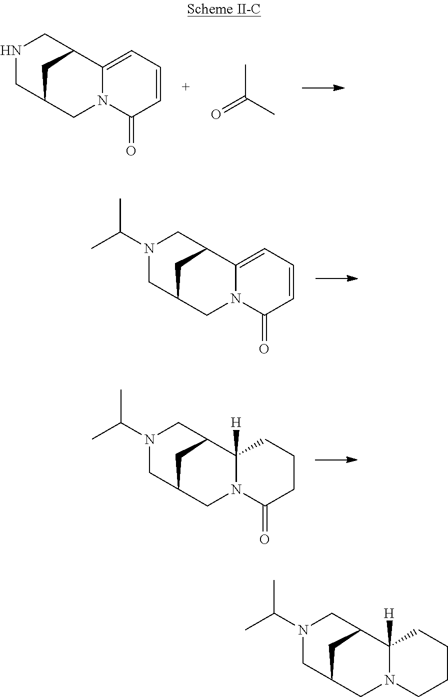

The ligands listed below were synthesized according to Schemes II-A-II-C.

n-Propyl Analog

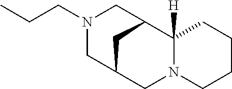

¹H NMR (500 MHz, DMSO) δ 2.93 (d, J=11.0 Hz, 1H), 2.74 (ddd, J=10.5, 4.3, 2.0 Hz, 2H), 2.65 (d, J=11.1 Hz, 1H), 2.29-2.12 (m, 2H), 2.05-1.85 (m, 4H), 1.85-1.65 (m, 4H), 1.64-1.14 (m, 12H).

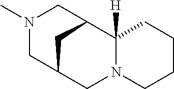

¹H NMR (500 MHz, CDCl₃) δ 2.99 (d, J=xx Hz, 2H), 2.89 (m, 2H), 2.24 (d, J=xx Hz, 1H), 2.16 (s, 3H), 1.97 (d, J=xx Hz, 1H), 1.91 (d, J=xx Hz, 1H), 1.81 (br. s, 1H), 1.78-1.63 (m, 4H), 1.62-1.43 (m, 5H), 1.36-1.21 (m, 2H).

n-Pentyl Analog

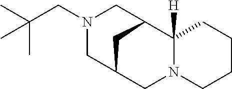

¹H NMR (500 MHz, CDCl₃) δ 3.10-2.92 (d, J=15.1 Hz, 1H), 2.86 (d, J=10.5 Hz, 1H), 2.79 (t, J=15.3 Hz, 1H), 2.71 (t, J=13.9 Hz, 1H), 2.52 (br. s, 1H), 2.38 (br. s, 1H), 2.29 (br. s, 1H), 2.10-1.89 (m, 3H), 1.89-1.41 (m, 9H), 1.40-1.18 (m, 2H), 0.89 (t, J=7.6 Hz, 9H).

iso-Propyl Analog

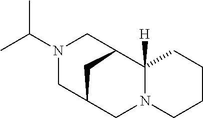

¹H NMR (500 MHz, CDCl₃) δ 3.01-2.54 (m, 6H), 2.32-2.09 (m, 2H), 1.97-1.66 (m, 4H), 1.66-1.33 (m, 6H), 1.33-1.18 (m, 2H), 1.05 (d, J=6.7 Hz, 3H), 0.93-0.83 (m, 3H).

The ligands of formula III were used to prepare a compound of formula I-1a. The results are summarized in Table 1.

TABLE 1

| | | Crude Product | | Recryst. Product | |
|---|---|---|---|---|---|
| Ligand | Yield | exo:endo | exo e.r. | exo:endo | exo e.r. |
| 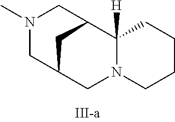 | 44% (recryst) | 92:8 | 90:10 | 97:3 | 99.6:0.4 |
| 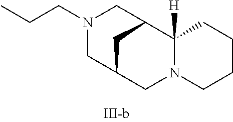 [SMILES: CCCN1C[C@@H]2C[C@H](C1)[C@@H]1CCCCN1C2] | 35% (recryst) | 92:8 | 88:12 | 97.5:2.5 | 100:0 |
| 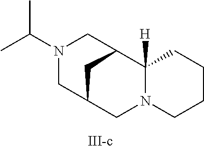 | | 94:6 | 87:13 | | |
| 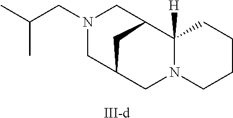 | 116% (crude) | 85:15 | 94:6 | | |
| 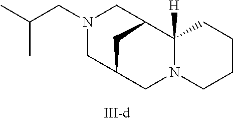 | 25% (recryst) | 93:7 | 91:9 | 98:2 | 100:0 |
| 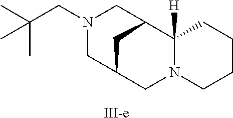 | | 88:12 | 63:37 | | |

* stirred for 5.5 hours before quenching

Scheme 3 depicts the reaction of a compound of formula 26 with a compound of formula I-1a to form a compound of formula 28.

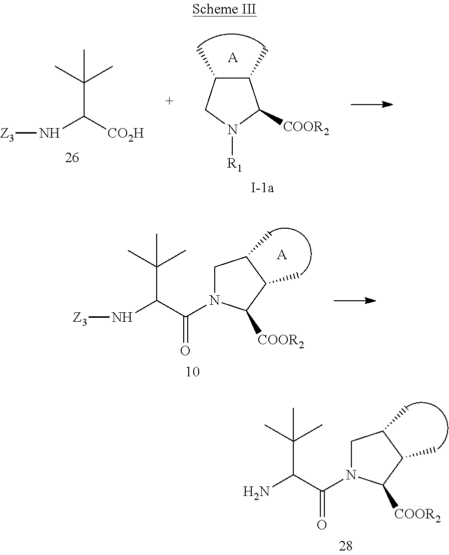

Referring to Scheme III, wherein R₁, R₂, Z₂, Z₃, and ring A are as defined previously, a bicyclic aminoester of formula I-1a, wherein R₂ is tert-butyl, is reacted with a protected amino acid of formula 26 (wherein Z₃ is an amine protecting group and can be removed under acidic, basic, or hydrogenating conditions different from those used for removing an R₂ protecting group) in the presence of a coupling reagent, to give an amide-ester of formula II. The protecting group Z₂ is removed from the amide-ester of formula 10 to give the amine-ester compound of formula 28.

In another aspect, compounds of formula 10 are intermediates in the synthesis of protease inhibitors according to Scheme IV.

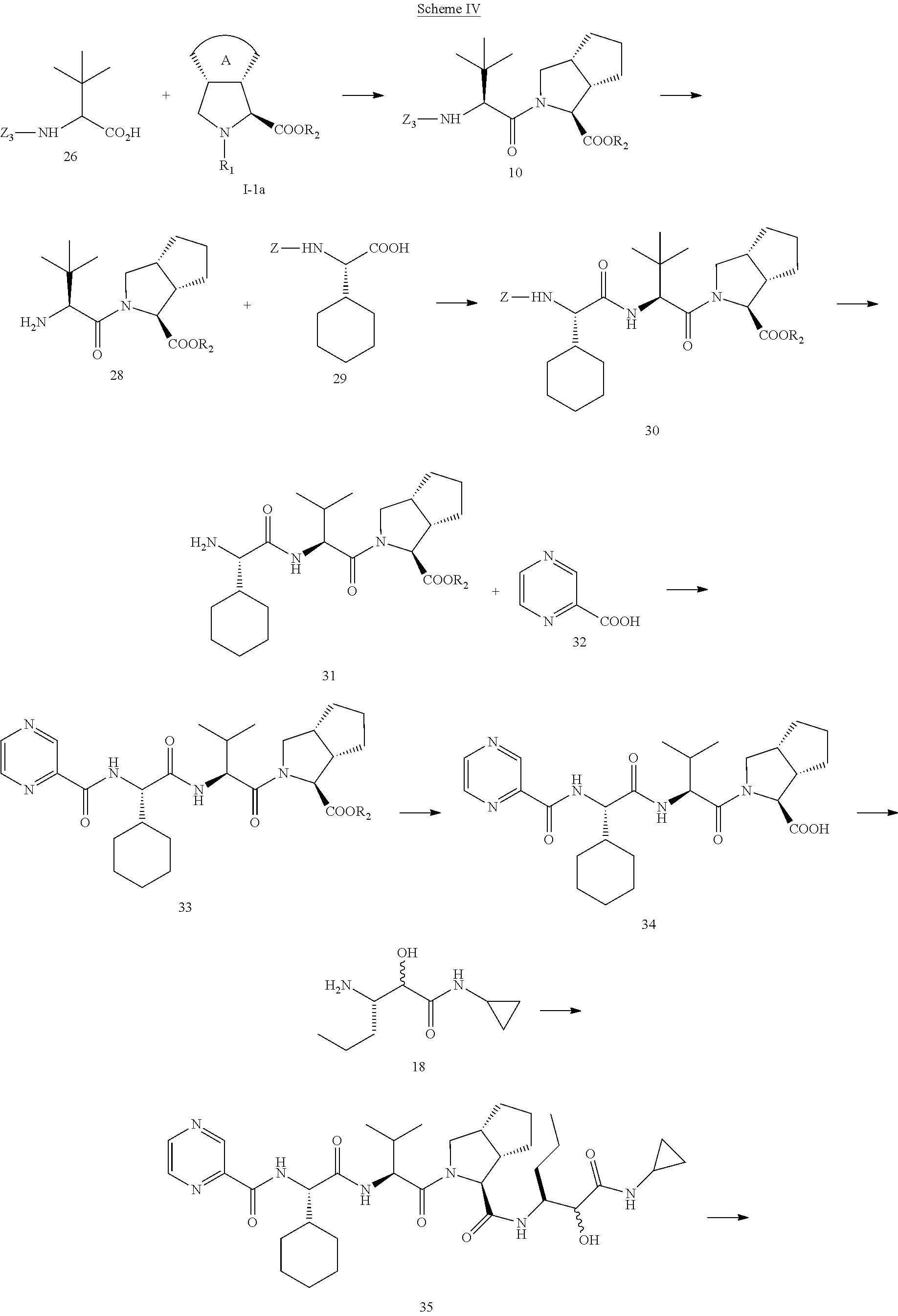

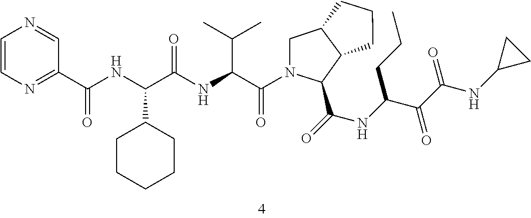 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)C)C(=O)C(=O)NC1CC1]

Scheme IV is disclosed in U.S. Pat. No. 7,776,887, the entire contents of which are incorporated herein by reference.

In Scheme IV, the bicyclic aminoester of formula I-1a, which can be prepared as described herein, wherein R₂ is tert-butyl, is reacted with a protected amino acid of formula 26 (wherein Z₃ is an amine protecting group and can be removed under acidic, basic, or hydrogenating conditions different from those used for removing the R₂ protecting group) in the presence of a coupling reagent, to give an amide-ester of formula 10. The protecting group Z₂ is removed from the compound of formula 10 to give the amine-ester compound of formula 28. Reaction of the amino-containing compound of formula 28 with the protected amino acid 29 in the presence of a coupling reagent gives a tripeptide of formula 30. Removing the protecting group Z in the tripeptide of formula 30 provides a free amino-tripeptide of formula 31. Reaction of the amino-tripeptide of formula 31 with the pyrazine-2-carboxylic acid of formula 32 in the presence of a coupling reagent yields the amide-tripeptide ester of formula 33. Hydrolysis of the ester of the amide-tripeptide ester of formula 33 provides the amido-tripeptide acid of formula 34. Reacting the amido-tripeptide acid of formula 34 with the amino-hydroxy amide of formula 18 in the presence of a coupling reagent gives the hydroxy-peptide of formula 35. In the final step, oxidation of the hydroxy group of the compound of formula 35 provides the compound of formula 4.

In another embodiment, the process of Scheme III can be scaled for large-scale production, e.g. in a manufacturing plant. Large scale production can, for example, be scaled to greater than 1000 kilos.

Although in parts of Schemes Ito IV, only a single isomer is illustrated for some of the compounds, the present invention is intended to include all stereoisomers of the compound.

The following non-limiting examples are set forth in order that this invention be more fully understood. These examples are for the purpose of illustration only and are not to be construed as limiting the scope of the invention in any way.

### EXAMPLES

### Example 1

### N-tert-butyloxycarbonyl-3-azabicyclo[3.3.0]octane (6)

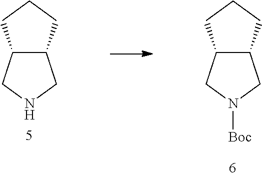 [SMILES: C1C[C@@H]2CNC[C@@H]2C1 ; CC(C)(C)OC(=O)N1C[C@H]2CCC[C@H]2C1]

### Method 1

Under nitrogen, 3-azabicyclo[3.3.0]nonane hydrochloride (100 g, 0.677 mol), potassium carbonate (187 g, 1.35 mol), MTBE (220 mL), and water (160 mL) were charged, with stirring, to a 2 L 3-necked round-bottom flask fitted with a mechanical stirrer, a 500 mL addition funnel, and a thermometer. The mixture was cooled to 14 to 16° C. Boc₂O (di-tert-butyl dicarbonate) (145 g, 0.644 mol) and MTBE (190 mL) was charged to a 500 mL Erlenmeyer flask. The mixture was stirred until dissolution was complete. The solution was poured into the addition funnel and added to the reaction mixture, keeping the reaction temperature below 25° C. Water (290 mL) was added to dissolve solids, and the mixture was stirred for 10 to 15 minutes. After removing the aqueous phase, the organic phase was washed with 5% aqueous NaHSO₄ (twice, 145 mL each), then water (145 mL). The organic phase was concentrated, and MTBE was added (1.3 L) to give a solution of the title compound in MTBE. See, e.g., R. Griot, Helv. Chim. Acta., 42, 67 (1959).

### Method 2

A solution of potassium carbonate (187 g, 1.35 mol) in water (160 mL) was added to a mixture of 3-azabicyclo[3.3.0]octane hydrochloride (100 g, 0.677 mol) and MTBE (220 mL), and the resulting mixture was cooled to 14 to 16° C. A solution of Boc₂O (145 g, 0.644 mol) in MTBE (190 mL) was added while maintaining a temperature below 35° C. After the addition, the mixture was stirred for 1 hour, then filtered. The solids were washed with MTBE (50 mL). The phases were then separated, and the organic phase was washed with 5% aqueous NaHSO₄ (twice, 145 mL each) and water (145 mL). It was then concentrated to 300 mL under vacuum. MTBE (300 mL) was added, and the mixture was concentrated to reduce the water concentration to less than 550 ppm. The concentrate was diluted with MTBE (400 mL) to provide a solution of the title compound in MTBE.

### Example 2

### Ligand Synthesis (Compound III)

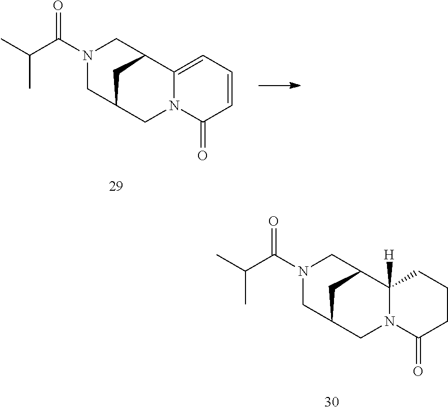 [SMILES: CC(C)C(=O)N1C[C@@H]2C[C@H](C1)c1cccc(=O)n1C2 ; CC(C)C(=O)N1C[C@@H]2C[C@H](C1)[C@@H]1CCCC(=O)N1C2]

To the starting material, compound 29 (13.68 g, 52.55 mmol), was added MeOH (273.6 mL), then PtO₂ (596.8 mg, 2.628 mmol). The reaction vessel was evacuated and vented with H₂ (three times). The mixture was stirred at 20 to 22° C. for 16 hours, when GC analysis showed 90% reaction completion. H₂ was recharged, and the mixture was stirred for 4 hours, when GC analysis showed 99% reaction completion. The mixture was filtered through a pad of Celite®, and the Celite® was rinsed with MeOH. The filtrate was concentrated at reduced pressure to give 11.00 g of the crude product as a thick brown oil. The product, compound 30, was used in the next step without purification.

¹H-NMR (d₆-DMSO, 500 MHz): δ4.82 (d, 1H); 4.59 (d, 1H); 3.99 (d, 1H); 3.46 (m, 1H); 3.27 (d, 1H); 2.70 (m, 2H); 2.59 (d, 1H); 2.18-1.41 (m, 10H); 0.90 (d, 3H); 0.85 (d, 3H).

¹³C-NMR (d₆-DMSO, 125 MHz): 8174.27, 167.95, 58.62, 49.71, 41.48, 40.02, 32.70, 32.53, 32.34, 29.07, 27.69, 27.32, 19.53, 19.38, 19.16.

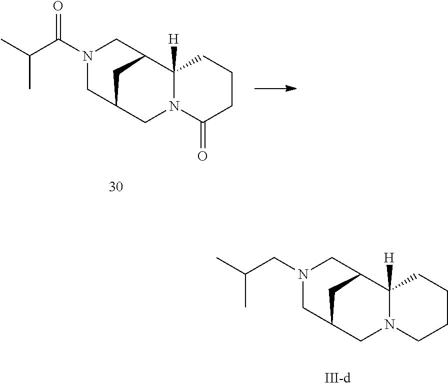 [SMILES: CC(C)C(=O)N1C[C@@H]2C[C@H](C1)[C@@H]1CCCC(=O)N1C2 ; CC(C)CN1C[C@@H]2C[C@H](C1)[C@@H]1CCCCN1C2]

To the starting material, compound 30 (11.00 g, 41.61 mmol), was added THF (198.0 mL), followed by LiAlH₄ (9.477 g, 10.33 mL, 249.7 mmol). The mixture was warmed to reflux for 21 hours, when GC analysis showed that the starting material was consumed. The mixture was cooled to 5 to 10° C., and MTBE (100 mL) was added. The mixture was carefully quenched with saturated a Na₂SO₄ solution. The reaction temperature rose to 44 to 45° C. during the quench. The slurry was filtered through a pad of Celite® and rinsed with a 9 to 1 mixture of DCM to MeOH (1 L). The solution was concentrated at reduced pressure, and the residue was suspended in EtOAc. The mixture was filtered again through Celite®, and the filtrate was concentrated at reduced pressure to give a light brown oil. The crude product was purified by Kugelrohr distillation (140 to 180° C.) to give 7.56 g of product, Compound III-d (77% yield).

¹H-NMR (d₆-DMSO, 500 MHz): δ2.92 (d, 1H); 2.78 (m, 3H); 2.31 (br. d, 1H); 2.20 (br. d, 1H); 2.05 (m, 2H); 1.87 (m, 2H); 1.76 (m, 4H); 1.62-1.40 (m. 6H); 1.26 (m, 2H); 0.90 (d, 3H); 0.85 (d, 3H).

¹³C-NMR (d₆-DMSO, 125 MHz): δ67.37, 65.90, 61.02, 58.93, 57.13, 53.55, 35.17, 34.05, 30.75, 30.63, 26.14, 25.62, 25.37, 21.05, 20.62.

### Example 3

### (S)-1,2,3,4-tetrahydronaphthalen-1-aminium (1S,3aR,6aS)-2-(tert-butoxycarbonyl)octahydrocyclopenta[c]pyrrole-1-carboxylic acid (9a)

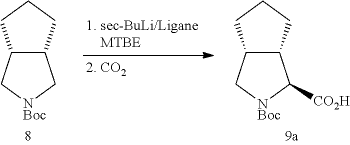

### Method 1

To a ligand (e.g., compound III-d (5.68 g, 24.03 mmol)) was added MTBE (39.05 mL) and compound 8 (3.905 g, 18.48 mmol). The solution was cooled to −75 to −70° C. sec-BuLi (15.25 g, 20.33 mL of 1.0 M, 20.33 mmol) was added, and the reaction temperature was kept below −65° C. The mixture was stirred for 5.5 hours at −75 to −70° C. CO₂ gas was bubbled into the reaction mixture, keeping the reaction temperature below −65° C. The solution was warmed to 22 to 25° C. and quenched with saturated NaHSO₄. The phases were separated, and the organic phase was washed with saturated NaHSO₄. The aqueous phase was extracted with MTBE (once, 40 mL).

The organic phase was extracted with 2N sodium hydroxide solution (twice, 40 mL). The pH of the combined aqueous phases was adjusted to about 2 to 3, and the aqueous phase was extracted with MTBE (twice, 40 mL). The MTBE solution was dried over Na₂SO₄ and filtered, and the solvent was removed at reduced pressure. The remaining oil (3.63 g) was dissolved into 11 mL of MTBE (3 vol), 11 mL of heptane was added, and the solution was stirred for 1 hour to give a white slurry. The mixture was cooled to 5 to 10° C. and stirred for 1 hour. Heptane (11 mL) was added, and the mixture was stirred for another 2 hours. The slurry was filtered, and the solids were rinsed with heptane. The white solid was dried to give 1.19 g of purified product, compound 9a (25% yield).

HPLC results: e.r. of exo isomer: 100:0; d.r. of exo:endo: 97.6:2.4.

### Example 4

### (1S,3aR,6aS)-tert-butyl 24(S)-2-(benzyloxycarbonylamino)-3,3-dimethylbutanoyl)octahydrocyclopenta[c]pyrrole-1-carboxylate (27)

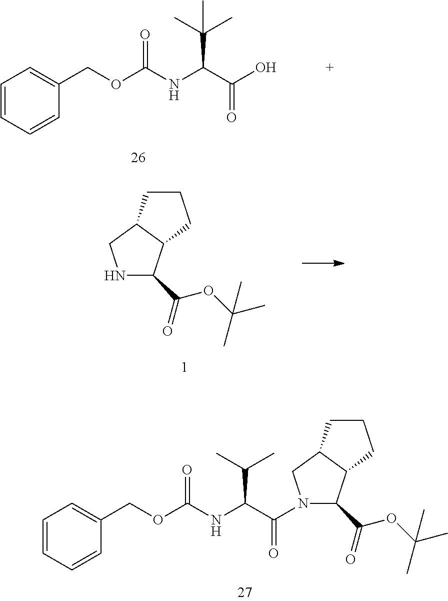 [SMILES: CC(C)(C)[C@H](NC(=O)OCc1ccccc1)C(=O)O ; CC(C)(C)OC(=O)[C@H]1NC[C@@H]2CCC[C@@H]21 ; CC(C)[C@H](NC(=O)OCc1ccccc1)C(=O)N1C[C@@H]2CCC[C@@H]2[C@H]1C(=O)OC(C)(C)C]

### Method 1

A 3 L 3-neck round bottom flask equipped with an overhead stirrer, condenser, thermocouple, and nitrogen outlet was purged with nitrogen for several minutes. In a separate flask, sulfuric acid (46.2 mL, 0.867 mol) was diluted with 442 mL of water. The solution was allowed to cool slightly. Cbz-L-tert-Leucine dicyclohexylamine salt (330.0 g, 0.739 mol) was charged to the reaction flask. MTBE (1620 mL) was added to the reactor, and the mixture was stirred to suspend the salt. The sulfuric acid solution prepared above was added to the reactor over about 10 minutes, keeping the temperature at 20±5° C. The mixture was stirred at room temperature for approximately 1 hour, then diluted slowly with water (455 mL). Agitation was stopped, and the layers were allowed to settle. The aqueous phase was withdrawn to provide 1100 mL colorless solution of pH 1. To the organic phase remaining in the flask was charged additional water (200 mL). The mixture was stirred at room temperature for approximately 1 hour. Agitation was stopped, and the layers were allowed to settle. The aqueous phase was withdrawn to provide 500 mL colorless solution of pH 2. The organic phase was heated to about 35° C., diluted with DMF (300 mL), and concentrated at reduced pressure to the point at which distillation slowed significantly, leaving about 500 mL of concentrate. The concentrate was transferred without rinsing to a 1 L Schott bottle. The concentrate, a clear colorless solution, weighed 511.6 g. Based on solution assay analysis and the solution weight, the solution contained 187.2 g (0.706 mol) of carboxybenzyl-L-tert-Leucine (Cbz-L-tert-Leucine).

To a 5 L 4-neck round bottom flask equipped with an overhead stirrer, thermocouple, addition funnel and nitrogen inlet were charged HOBT.H₂O (103.73 g, 0.678 mol, 1.20 molar eq.), EDC.HCl (129.48 g, 0.675 mol, 1.20 molar eq.), and DMF (480 mL). The slurry was cooled to 0 to 5° C. A 36.6 weight percent solution of the acid of Cbz-L-tert-Leucine in DMF (491.3 g, 0.745 mol, 1.32 molar eq.) was added over 47 minutes to the reaction mixture, while keeping the temperature at 0 to 5° C. The reaction mixture was stirred for 1 hour and 27 minutes. A solution of 3-azabicyclo(3.3.0)octane-2-carboxylic acid-tert-butyl ester in isopropyl acetate (28.8 weight percent, 414.3 g, 0.564 mol) was added over 53 minutes, while keeping the reaction temperature at 0 to 5.1° C. The reaction mixture was warmed to 20±5° C. over about 1 hour. 4-Methylmorpholine (34.29 g, 0.339 mol, 0.60 molar eq.) was added over 5 minutes. The reaction mixture was agitated for 16 hours, and then isopropyl acetate (980 mL) was added to the reaction solution. A solution of histamine.2HCl (41.58 g, 0.226 mol, 0.40 molar eq.) in water (53.02 g) was added to the reaction mixture within 4 minutes, followed by 4-methylmorpholine (45.69 g, 0.45 mol, 0.80 molar eq.). The reaction mixture was sampled after 3.5 hours. Water (758 mL) was added, and the mixture was stirred for about 20 minutes, then allowed to settle for 11 minutes. The phases were separated. The aqueous phase was extracted with isopropyl acetate (716 mL), and the organic phases were combined. 1 N aqueous hydrochloric acid was prepared by adding 37 weight percent hydrochloric acid (128.3 mL) to water (1435 ml). The organic phase was washed for about 20 minutes with the 1 N hydrochloric acid. A 10 weight percent aqueous potassium carbonate solution was prepared by dissolving potassium carbonate (171 g, 1.23 mol, 2.19 molar eq.) in water (1540 mL). The organic phase was washed with the 10 weight percent aqueous potassium carbonate solution for about 20 minutes. The final clear, pale yellow organic solution (1862.1 g), was sampled and submitted for solution assay. Based on the solution assay and the weight of the solution, the solution contained 238.3 g (0.520 mol) of product of the title compound.

¹H NMR (DMSO-d₆, 500 MHz): δ 7.37 ppm (5H, s), 7.25-7.33 ppm (1H, m), 5.03 ppm (2H, s), 4.17 ppm (1H, d), 3.98 ppm (1H, d), 3.67-3.75 ppm (2H, m), 2.62-2.74 ppm (1H, m), 2.48-2.56 ppm (1H, m), 1.72-1.89 ppm (2H, m), 1.60-1.69 ppm (1H, m), 1.45-1.58 ppm (2H, m), 1.38 ppm (9H, s), 1.36-1.42 ppm (1H, m), 0.97 ppm (9H, s).

### Method 2

A solution of potassium carbonate (73.3 g) in water (220 mL) was added to a suspension of (1S,2S,5R)3-azabicyclo[3.3.0]octane-2-carboxylic-ten-butylester-oxalate (80.0 g) in isopropyl acetate (400 mL) while maintaining a temperature of about 20° C. The mixture was stirred for 0.5 hours, the phases were separated, and the organic phase was washed with 25 weight percent aqueous potassium carbonate (80 mL) to give a solution of the free base. In a separate flask, aqueous sulfuric acid (400 mL, 0.863 M) was added to a suspension of Cbz-tert-leucine dicyclohexylamine salt (118.4 g) in tert-butylmethyl ether (640 mL) while maintaining a temperature of about 20° C. The mixture was stirred for 0.5 hours, the phases were separated, and the organic phase was washed with water (200 mL). The phase were separated, and N-methylmorpholine (80 mL) was added to the organic phase, which was concentrated at reduced pressure at 40° C. to 80 mL to give the free acid as a solution in N-methymorpholine. This solution was added to a mixture of EDC.HCl (50.8 g) and HOBt hydrate (40.6 g) in N-methylmorpholine (280 mL) at 0 to 10° C. The mixture was stirred for 1 hour at about 5° C. The solution of 3-azabicyclo[3.3.0]octane-2-carboxylic, tert-butylester from above was added at 0 to 20° C., followed by N-methylmorpholine (32 mL). The mixture was stirred for 6 hours, then diluted with isopropyl acetate (600 mL) followed by 1 N hydrochloric acid (400 mL). After stirring 0.5 hours, the phases were separated and the organic phase was washed with 25 weight percent aqueous potassium carbonate (400 mL) and water (80 mL). The mixture was stirred for about 1 hour, and the phases were separated to give a solution of the title compound in isopropyl acetate.

### Method 3

(1S,2S,5R)3-azabicyclo[3.3.0]octane-2-carboxylic-tert-butylester-oxalate (1.0 eq.) was suspended in isopropyl acetate (6 vol.), and a solution of potassium carbonate (3.0 eq.) in water (3.5 vol.) was added at 20 to 25° C. The mixture was stirred for 3 hours, then the phases were separated. The organic phase was washed with water (2 vol.).

Cbz-tert-leucine dicyclohexylamine salt (1.05 eq.) was suspended in isopropyl acetate (6 vol.), and sulfuric acid (1.3 eq.) in water (5 vol.) was added at 20 to 25° C. The mixture was stirred for 30 minutes, the phases were separated, and the organic phase was washed with water (2 times, 2.5 vol.).

The two solutions from above were combined and then cooled to 0 to 5° C. HOBt hydrate (1.1 eq.) and EDC (1.1 eq.) were suspended in the mixture, and the mixture was stirred for 6 hours. The mixture was washed with water (5 vol.), and the resulting organic phase was treated with L-lysine (1 eq.) and N-methylmorpholine (2 eq.) at 20 to 25° C. to destroy excess activated ester. The mixture was then washed with 5 percent potassium carbonate (5 vol.), 1 N hydrochloric acid (5 vol.), 5 percent potassium carbonate (5 vol.), and water (twice, 5 vol.) to give a solution of the title compound in isopropyl acetate.

### Example 5

### (1S,3aR,6aS)-tert-butyl 2-((S)-2-amino-3,3-dimethylbutanoyl)-octahydrocyclopenta[c]pyrrole-1-carboxylate (28)

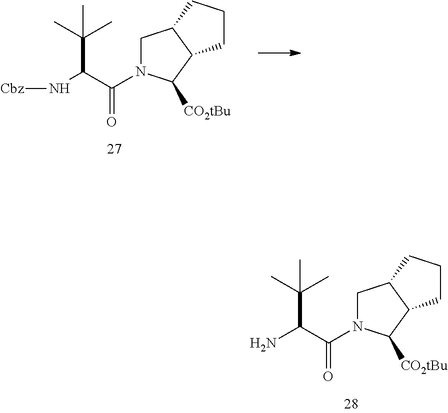 [SMILES: CC(C)(C)OC(=O)[C@@H]1[C@H]2CCC[C@H]2CN1C(=O)[C@@H](NC(=O)OCc1ccccc1)C(C)(C)C ; CC(C)(C)OC(=O)[C@@H]1[C@H]2CCC[C@H]2CN1C(=O)[C@@H](N)C(C)(C)C]

### Method 1

A 1 L Buchi hydrogenator was purged with nitrogen three times. A 307.8 g portion of a 12.8 weight percent solution of (1S,3aR,6aS)-tert-butyl 2-((S)-2-(benzyloxycarbonylamino)-3,3-dimethylbutanoyl)octahydrocyclopenta[c]pyrrole-1-carboxylate (as prepared by the method of Example 6, Method 1) in isopropyl acetate (39.39 g, 0.086 mol) was charged to the reactor. Isopropyl acetate (100 mL) was added to the reactor. A slurry of 50% water and wet 20% Pd(OH)₂/carbon (3.97 g) in isopropyl acetate (168 mL) was prepared and charged to the reactor, and agitation was started. The reactor was pressurized to 30 psig with nitrogen gas and vented down to atmospheric pressure. This was repeated twice. Then, the reactor was pressurized to 30 psig with hydrogen and vented down to atmospheric pressure. This was repeated twice. The reactor was pressurized to 30 psig with hydrogen and stirred at ambient temperature for 1 hour. The mixture was filtered using a Buchner funnel with a Whatman #1 filter paper to remove the catalyst. The filter cake was washed with isopropyl acetate (80 mL). The procedure was repeated twice more using 617 g and 290.6 g of the 12.8 weight percent solution of the starting compound. The material from the three hydrogenations were combined and distilled at reduced pressure (28 torr). The resultant solution (468.68 g) was assayed for the title compound.

¹H NMR (DMSO-d₆, 500 MHz): δ 3.96 ppm (1H, d), 3.67 ppm (1H, dd), 3.53 ppm (1H, dd), 3.19 ppm (1H, s), 2.66-2.75 ppm (1H, m), 2.49-2.53 ppm (1H, m), 1.75-1.92 ppm (2H, m), 1.66-1.74 ppm (1H, m), 1.48-1.60 ppm (4H, m), 1.38 ppm (9H, s), 1.36-1.42 ppm (1H, m), 0.91 ppm (9H, s)

### Method 2

The solution of the Cbz derivative 27 from Example 6, Method 2, was added to 20% Pd(OH)₂/water (50%, 12.2 g) in a hydrogenation apparatus. The apparatus was pressurized to 30 psi with hydrogen, then stirred for 2 hours at about 20° C. The mixture was filtered to remove the catalyst, and the filter cake washed with isopropyl acetate (160 mL). The combined filtrates were evaporated with about 4 volumes of heptane at 40° C. 2 to 3 times to remove the isopropyl acetate. The resultant slurry was cooled to 0° C. and filtered, and the product was dried at reduced pressure to give the title compound.

### Method 3

A solution of (1S,3aR,6aS)-tert-butyl 2-((S)-2-amino-3,3-dimethylbutanoyl)-octahydrocyclopenta[c]pyrrole-1-carboxylate in isopropyl acetate from Example 6, Method 3, was added to 20% Pd(OH)₂ (2 weight percent loading, 50 percent wet) and the mixture was hydrogenated at 2 bar and 20 to 25° C. for 2 hours. The catalyst was removed by filtration and washed with isopropyl acetate (2 vol.). The filtrate was concentrated to 10 vol. at reduced pressure at 40° C. to give a solution of the title compound in isopropyl acetate.

While we have presented a number of embodiments of this invention, it is apparent that our basic construction can be altered to provide other embodiments which utilize the compounds and methods of this invention. Therefore, it will be appreciated that the scope of this invention is to be defined by the appended claims rather than by the specific embodiments which have been represented by way of example.

What is claimed is:

1. A process for preparing enantioselectively a compound of formula I-1a or I-1b: 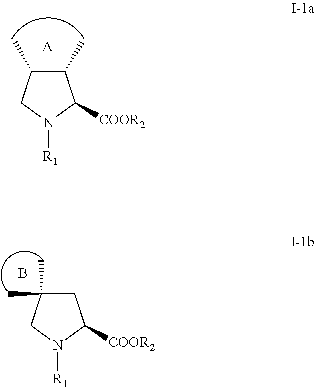
over a compound of formulas I-2-I-7: 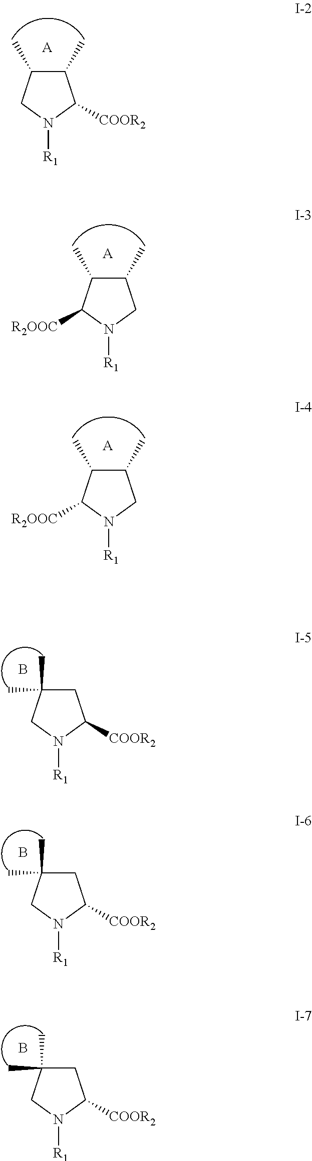
comprising the step of carboxylating a compound of formulas II-a or II-b: 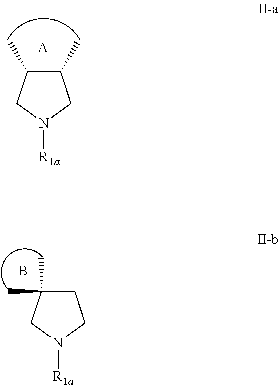
in the presence of a compound of formula III: 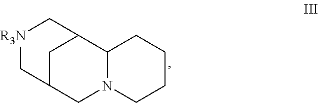
wherein:
Ring A is a C₃₋₁₂ cycloaliphatic ring;
Ring B is a C₃₋₁₂ heterocycloaliphatic ring containing additional 0 to 2 heteroatoms, each independently selected from O, N, and S, wherein ring B is optionally substituted with 0 to 4 groups, each independently selected from alkyl, halo, alkoxy, aryl, and hydroxy;
R₁ is H or a protecting group;
R₁ₐ is a protecting group;
R₂ is H, a protecting group, or C₁₋₁₂ aliphatic; and
R₃ is C₁₋₁₂ aliphatic or a protecting group.

2. The process of claim 1, wherein ring A is C₃₋₆ cycloaliphatic.

3. The process of claims 1-2, wherein ring A is cyclopropyl.

4. The process of claims 1-2, wherein ring A is cyclopentyl.

5. The process of claims 1-2, wherein ring A is 1,1-dimethylcyclopropyl.

6. The process of claims 1-2, wherein ring A is:

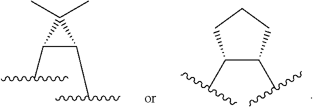

7. The process of claim 6, wherein ring A is

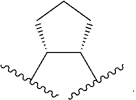 [SMILES: *[C@@H]1CCC[C@@H]1*]

8. The process of claim 6, wherein ring A is

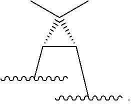 [SMILES: *[C@@H]1[C@H](*)C1(C)C]

9. The process of claim 1, wherein ring B is a 5-membered heterocyclic ring.

10. The process of claim 1, wherein ring B is an optionally substituted ring of the following formula:

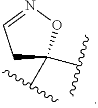

11. The process of claim 1, wherein ring B is substituted with an aryl ring optionally substituted with 0 to 4 groups, each independently selected from alkyl, halo, alkoxy, and hydroxyl.

12. The process of claim 11, wherein the aryl ring is phenyl.

13. The process of claim 11, wherein the aryl ring is

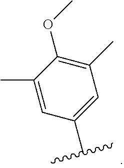

14. The process of claims 9-13, wherein ring B is the following:

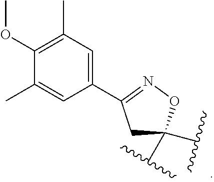

15. The process of claims 1-14, wherein R₂ is H.

16. The process of claims 1-14, wherein R₂ is C₁₋₁₂ aliphatic.

17. The process of claims 1-14, wherein R₂ is tert-butyl.

18. The process of claims 1-17, wherein the step of carboxylating a compound of formula II-a or II-b is in presence of the compound of formula III-a:

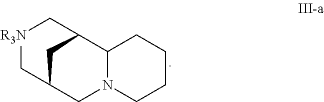

19. The process of claim 18, wherein the step of carboxylating a compound of formula II-a or II-b is in presence of the compound of formula III-b:

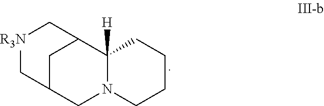

20. The process of claim 18, wherein the step of carboxylating a compound of formula II-a or II-b is in presence of the compound of formula III-c:

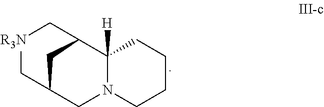

21. The process of claims 18-20, wherein R₃ is C₃₋₁₂ aliphatic.

22. The process of claims 18-20, wherein R₃ is a cycloaliphatic.

23. The process of claims 18-20, wherein R₃ is C₁₋₆ aliphatic.

24. The process of claims 18-20, wherein R₃ is C₁₋₆ alkyl.

25. The process of claims 18-20, wherein R₃ is selected from methyl, ethyl, n-propyl, iso-propyl, iso-butyl, n-butyl, n-pentyl, and iso-pentyl.

26. The process of claims 1-20, wherein R₃ is iso-butyl.

27. The process of claims 1-26, wherein R₁ₐ is tert-butyl carbamate (Boc).

28. The process of claims 1-27, wherein the carboxylation step includes treating a compound of formula II with carbon dioxide and a lithium base in the presence of an aprotic solvent.

29. The process of claim 28, wherein the aprotic solvent is toluene, ethyl acetate, benzene, and methyl tert-butyl ether.

30. The process of claim 28, wherein the aprotic solvent is methyl tert-butyl ether.

31. The process of claim 28, wherein the lithium base is sec-butyl lithium.

32. The process of claims 1-31, wherein the combined weight percent in a mixture comprising compounds of formula I-1a and I-3 (the exo-isomers), and compounds of formula I-2 and I-4, (the endo-isomers), is 100 weight percent.

33. The process of claim 32, wherein the exo/endo ratio is at least 60 to 40.

34. The process of claim 32, wherein the exo/endo ratio is at least 80 to 20.

35. The process of claim 32, wherein the exo/endo ratio is at least 90 to 10.

36. The process of claim 32, wherein the exo/endo ratio is at least 95 to 5.

37. The process of claim 32, wherein the exo/endo ratio is at least 97 to 3.

38. The process of claims 32-37 further comprising removing portion of the compounds of formula I-2 and/or I-4 from the product mixture.

39. The process of claim 38, further comprising crystallizing the compound of formula I-1a or I-1b.

40. The process of claim 38, further comprising recrystallizing the compound of formula I-1a or I-1b.

41. The process of claims 38-40, wherein the ratio of the weight percent of I-1a to I-3 is at least 60 to 40.

42. The process of claims 38-40, wherein the ratio of the weight percent of I-1a to I-3 is at least 80 to 20.

43. The process of claims 38-40, wherein the ratio of the weight percent of I-1a to I-3 is at least 90 to 10.

44. The process of claims 38-40, wherein the ratio of the weight percent of I-1a to I-3 is at least 95 to 5.

45. The process of claims 38-40, wherein the ratio of the weight percent of I-1a to I-3 is at least 99 to 1.

46. The process of claims 38-40, wherein the ratio of the weight percent of I-1a to I-3 is at least 99.6 to 0.4.

47. The process of claims 38-40, wherein the ratio of the weight percent of I-1a to I-3 is 100 to 0.

48. A process for preparing a compound of formula 10: 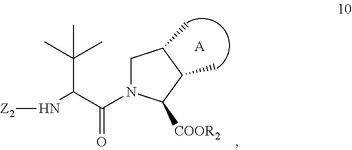
wherein Z₂ is H or a protecting group, and R₂ is H, a protecting group, or C₁₋₁₂ aliphatic, comprising the steps of:
i) providing a compound of formula II-a: 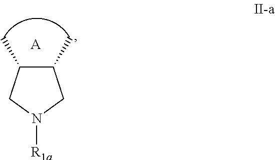
wherein R₁ₐ is a protecting group, and ring A is C₃₋₁₂ cycloaliphatic;
ii) forming a 2-anion of the compound of formula II-a in the presence of a compound of formula III: 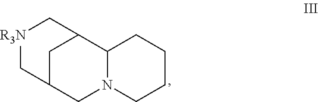
wherein R₃ is C₁₋₁₂ aliphatic or a protecting group;
iii) treating the anion of step ii) with carbon dioxide to produce enantioselectively a compound of formula I-1a; and
iv) reacting the compound of formula I-1a with a compound of formula 26, 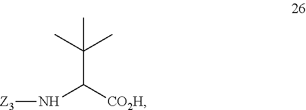
wherein Z₃ is a protecting group, in the presence of a coupling reagent.

49. The process of claim 48, wherein R₃ is ten-butyl.

50. The process of claim 48 or 49, wherein the compound of formula III is formula III-a:

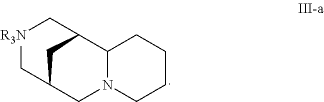

51. The process of claim 48, wherein the compound of formula III is formula III-b:

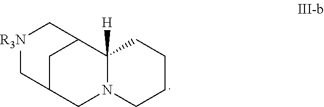

52. The process of claim 48, wherein the compound of formula III is formula III-c:

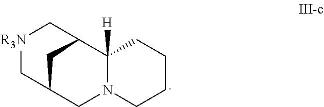

53. The process of claims 50-52, wherein R₃ is C₃₋₁₂ aliphatic.

54. The process of claims 50-52, wherein R₃ is a cycloaliphatic.

55. The process of claims 50-52, wherein R₃ is C₁₋₆ aliphatic.

56. The process of claims 50-52, wherein R₃ is C₁₋₆ alkyl.

57. The process of claims 50-52, wherein R₃ is methyl, ethyl, n-propyl, iso-propyl, iso-butyl, n-butyl, n-pentyl, or iso-pentyl.

58. The process of claims 50-52, wherein R₃ is iso-butyl.

59. The process of claims 48-58, wherein ring A is:

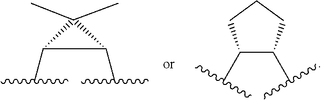

60. The process of claim 59, wherein ring A is

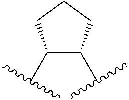 [SMILES: *[C@@H]1CCC[C@@H]1*]

61. The process of claims 48-60, wherein the compound of formula 26 is formula 26-b:

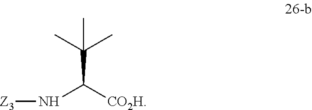

62. The process of claims 48-61, wherein the compound of formula 10 is formula 10-a:

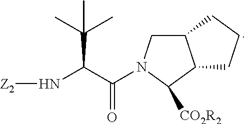

63. The process of claims 48-62, wherein the compound of formula 10 is formula 10-b:

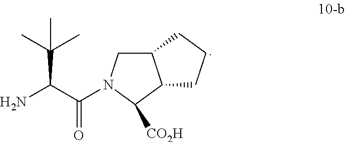 [SMILES: CC(C)(C)[C@H](N)C(=O)N1C[C@@H]2CCC[C@@H]2[C@H]1C(=O)O]

64. The process of claim 59, wherein ring A is

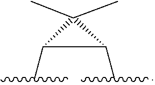

65. The process of claims 48-59 and 64, wherein the compound of formula 26 is formula 26-b:

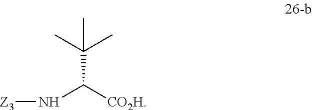

66. The process of claims 48-62, wherein the compound of formula 10 is formula 10-c:

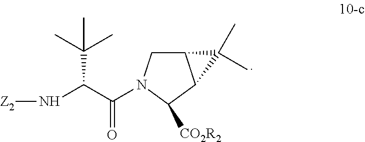

67. The process of claims 48-62, wherein Z₂ is H, and R₂ is tert-butyl.

68. A process for preparing compounds of formula 4: 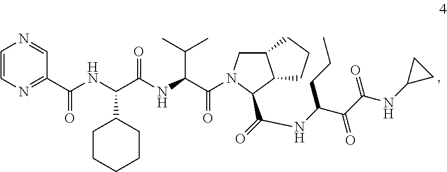 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)C)C(=O)C(=O)NC1CC1]
comprising the steps of:
i) reacting a compound of formula II-a with a base and CO₂ in the presence of a compound of formula III to prepare a compound of formula I-1a;
ii) reacting the a compound of formula I-1a with a compound of formula 26 in the presence of a coupling reagent to form a compound of formula 10;
iii) removing Z₂ from the compound of formula 10 to give a compound of formula 28: 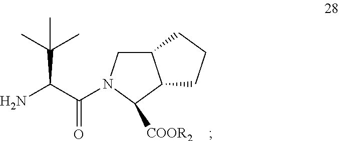
iv) reacting the compound of formula 28 with a compound of formula 29: 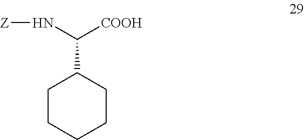
in the presence of a coupling reagent to give a compound of formula 30: 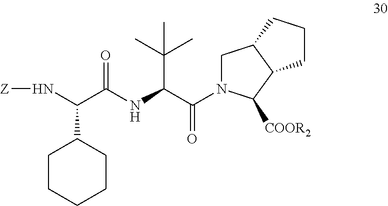
wherein Z is an amine protecting-group;
v) removing the protecting group Z in the compound of formula 30 to give a compound of formula 31: 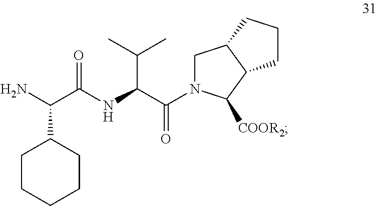
vi) reacting the compound of formula 31 with a compound of formula 32: 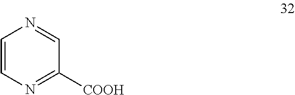
in the presence of a coupling reagent to give a compound of formula 33: 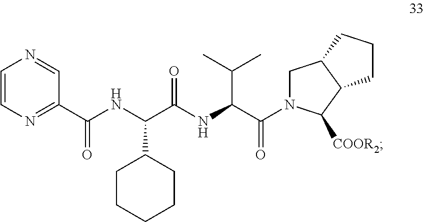
vii) hydrolyzing the ester of the compound of formula 33 to give a compound of formula 34: 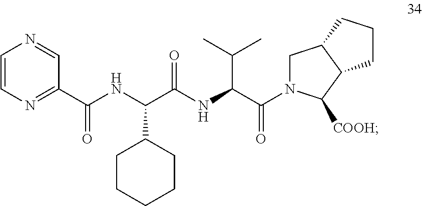 [SMILES: CC(C)[C@H](NC(=O)[C@@H](NC(=O)c1cnccn1)C1CCCCC1)C(=O)N1C[C@@H]2CCC[C@@H]2[C@H]1C(=O)O]
viii) reacting the compound of formula 34 with a compound of formula 18: 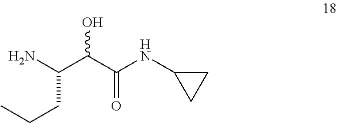
in the presence of a coupling reagent to give a compound of formula 35: 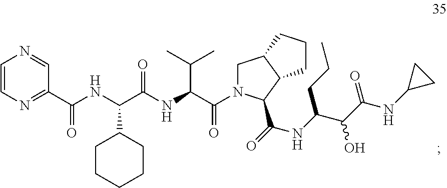 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)C)C(O)C(=O)NC1CC1]
ix) oxidizing the compound of formula 35 to give the compound of formula 4.

69. The process of claim 68, wherein the process is scaled for large scale production.

70. A compound of the formula I-1a(1) made by the process of claims 1-47: 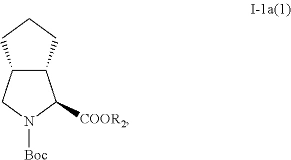
wherein R₂ is H, a protecting group, or C₁₋₁₂ aliphatic.

71. A compound of the formula I-1a(2) made by the process of claims 1-47:

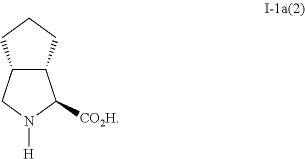 [SMILES: O=C(O)[C@H]1NC[C@@H]2CCC[C@@H]21]

72. A compound of the formula I-1a(3) made by the process of claims 1-47: 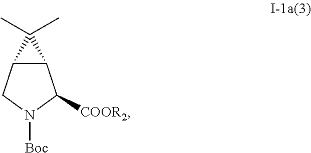
wherein R₂ is H, a protecting group, or C₁₋₁₂ aliphatic.

73. A compound of the formula I-1a(4) made by the process of claims 1-47:

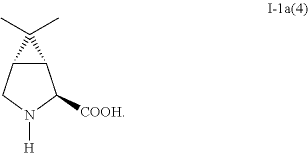

74. A compound of the formula 10-a made by the process of any one of claims 48-63:

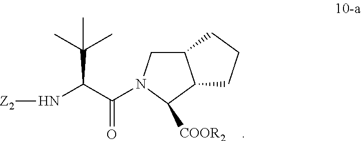

75. The compound of claim 73, wherein Z₂ is H, and R₂ is tert-butyl.

76. A compound of the formula 10-c made by the process of claims 48-59 and 64-67:

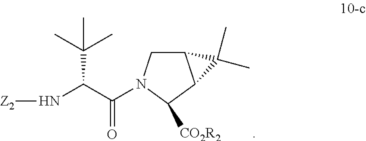

77. The compound of claim 76, wherein Z is H, and R₂ is tert-butyl.
